(19) United States
(12) Patent Application Publication
(10) Pub. No.: US 2024/0287430 A1
(43) Pub. Date: Aug. 29, 2024

(54) Systems and Methods for Cellular Lumen Formation and Cellular Differentiation

(71) Applicant: The Regents of the University of California, Oakland, CA (US)

(72) Inventors: Sebastian Streichan, Goleta, CA (US); Eyal Karzbrun, Goleta, CA (US)

(73) Assignee: The Regents of the University of California, Oakland, CA (US)

(21) Appl. No.: 18/566,324

(22) PCT Filed: Jun. 2, 2022
(86) PCT No.: PCT/US2022/072735

§ 371 Date: Dec. 1, 2023

Related U.S. Application Data
(60) Provisional application No. 63/196,149, filed on Jun. 2, 2021.

(51) Int. Cl. C12M 1/12; C12N 5/0793

(52) U.S. Cl. CPC C12M 25/14; C12N 5/0619; C12N 2501/155; C12N 2513/00; C12N 2533/90

(57) ABSTRACT

Systems and methods for cellular luminal structure formation are provided. In some instances, a cellular luminal structure is formed utilizing a micropatterned matrix, biological cells in contact with the micropatterned matrix, and suspended matrix in media. In some instances, cellular luminal structure is utilized in conjunction with cellular differentiation.

### CROSS REFERENCE TO RELATED APPLICATIONS

This application is a national stage of PCT Patent Application No. PCT/US2022/072735 entitled “Systems and Methods for Cellular Lumen Formation and Cellular Differentiation” filed Jun. 2, 2022, which claims priority to U.S. Provisional Application Ser. No. 63/196,149, entitled “Systems and Methods for Cellular Lumen Formation and Cellular Differentiation,” filed Jun. 2, 2021, which are incorporated herein by reference in their entirety.

### STATEMENT REGARDING FEDERALLY SPONSORED RESEARCH OR DEVELOPMENT

This invention was made with Government support under contract R21 HD099598 awarded by the National Institutes of Health. The Government has certain rights in the invention.

### TECHNICAL FIELD

The disclosure provides description of systems and methods to form cellular lumens and for potent cell differentiation.

### BACKGROUND

Cell differentiation is a biological process from which less specialized cells become more specialized in a maturation process. In mammals, the zygote (i.e., the initial cell post conception) is the least differentiated cell, which provides genetic material for development of all cell types via cell differentiation. During development, a multitude of stem cells are formed which have the main function of dividing into more cells that will populate throughout the body and differentiate into mature cells to provide a particular function and thus forming a complex system of tissues and cell types.

Cell potency is the ability of a cell to divide and give rise to various differentiated cell types. Thus, in mammals, the zygote is the most potent cell as it gives rise to every differentiated cell in the body and placenta. Embryonic cells are termed “pluripotent,” meaning that the cells have the potential differentiate into any cell within a mammalian body. As cells mature, their potency is further limited. For example, an embryonic stem cell can divide and mature into a multipotent neural stem cell. A neural stem cell can then divide and further mature into a neuron or astrocyte (cells that provide brain function).

Brain and spinal cord development begins with the folding of the embryonic neural tissue into a tube (FIG. 1A). Defects in neural folding are one of the most common birth defects affecting 1:1000 pregnancies, which can result in severe disabilities and lethality shortly after birth. This highlights the need for understanding the cellular and tissue scale processes which drive neural tube folding in humans. To address the limited accessibility in human embryos, there has been an accelerated development of 3D human stem cell cultures (organoids) capable of recapitulating selected aspects of human organ formation. Organoids have greatly advanced our understanding of cell fate decisions during organ formation, yet they yield unreproducible and anatomically incorrect tissue shapes and cell-fate patterns (FIG. 1B). Additional organs-on-chip systems have been developed to apply stem-cells into scalable, controlled and functional tissues. However, the integration of stem-cells in complex microfluidic environments over-constrains their shape, and does not allow for self-organization as in embryonic development. Thus, new approaches are required in order to study human organ morphogenesis.

### SUMMARY OF THE DISCLOSURE

Many embodiments are directed to methods and systems of forming a cellular lumen utilizing cellular techniques. In many of these embodiments, a cellular lumen is formed having dome-like or semi-tubular shape. In various embodiments, a system comprising a micropatterned matrix, cells in contact with the micropatterned matrix, and suspend matrix in a medium is utilized to form a lumen. Various embodiments are also directed towards utilization of methods and systems of cellular differentiation in conjunction with cellular lumen formation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments and should not be construed as a complete recitation of the scope of the disclosure.

FIG. 1A provides a schematic of neural fold morphogenesis in a human embryo. Neural plate shown in red, non-neural ectoderm in blue, and neural crest (NC) in yellow.

FIG. 1B provides a schematic and images of conventional in vitro organoid formation in accordance with prior art. Uncontrolled initial conditions result in irregular morphogenesis observed in conventional organoids.

FIG. 2A provides a method for forming a three-dimensional lumen utilizing cultured cells in accordance with embodiments.

FIG. 2B provides a method for cellular differentiation utilizing a three-dimensional lumen formation of cultured cells in accordance with embodiments.

FIG. 3 provides a schematic of reproducible morphogenesis utilizing a three-dimensional lumen formation of cultured cells in accordance with embodiments.

FIG. 4 provides a schematic of a neural cell differentiation protocol to form a neural tube utilizing a three-dimensional cellular lumen in accordance with embodiments. Cells are seeded on micropatterns on Day 1. On day 2 neural induction media containing 5 μM of TGFβ-inhibitor SB-431542. On Day 3 cells transition into a 3D tissue containing a single lumen. On days 5-9 neural induction media is supplemented with 5 ng/mL BMP4 in addition to 5 μM of SB-431542. Neural and ectoderm cell fates are observed on day 6 and folding morphogenesis is observed during days 7-9.

FIG. 5 provides a schematic and results of micropattern geometry in control of three-dimensional stem-cell culture shape in accordance with an embodiment. The schematic shows shape-controlled ECM pattern deposited on glass surface. The results show seeding shape-controlled ECM pattern deposited on glass surface. The results further show adding 4% Matrigel to the media results in three-dimensional stem-cell cultures containing a single large lumen. The 3D shape is controlled by the micropattern geometry. Scale bar is 50 μm.

FIG. 6 provides a schematic and results of the reproducibility and scalability of three human pluripotent stem cells with single lumen in accordance with embodiments. (A) Scheme showing micropatterned hPSC and imaging plane. (B) Micropattern array of hPSCs before Matrigel addition (Day 2). The array contains dozens to hundreds of individual colonies. (C) Scheme showing micropatterned 3D hPSC after Matrigel addition. (D) Micropattern array of hPSCs after Matrigel addition (Day 3). (E) 3D hPSC with a single lumen forms with success rate greater than 90%. (F) Segmentation of single nuclei is used to count the number of cell in each sample. Each nucleus is labeled with a different color. (G) Total cell number in each sample scales linearly with pattern area while cell density remains invariant. Total n=300. Scale bar is 500 μm.

FIG. 7 provides a schematic and results showing three-dimensional human pluripotent stem cells form a pluripotent epithelium surrounding a single lumen in accordance with embodiments. (A) Scheme showing micropatterned hPSC. Cells represented in green over grey surface. (B) Vertical section of micropattern hPSC immunostained with pluripotency marker OCT4 (day 2). (C-E) Horizontal sections showing pluripotency markers NANOG, OCT4, and SOX2. (F) Horizontal section near colony top surface showing tight junction protein ZO1. (G) Scheme showing micropatterned 3D hPSC after Matrigel addition. (H) Vertical section of 3D hPSC immunostained with pluripotency marker OCT4 (day 3). (I-K) Horizontal sections showing pluripotency markers NANOG, OCT4, and SOX2. (L) Horizontal section through center of the 3D culture showing tight junction protein ZO1. Scale bar 50 μm.

FIG. 8 provides a schematic and results showing that three-dimensional human pluripotent stem cells form a molecularly isolated niche in accordance with embodiments. (A) Experimental design. hPSC reporter line endogenously expressed tight junction protein ZO1 tagged with GFP (AICS-0023) is used. The 3D culture is exposed to 10 kda dextran tagged with Texas Red fluorophore. (B) Horizontal sections show that tight junctions are localized to the inner surface facing the lumen (green). Dextran is visible outside the tissue, but the lumen is devoid of dextran (red). The formation of tight junctions, and the exclusion of Dextran from the inner lumen, suggest that molecules cannot freely diffuse between the media and the inner lumen. Thus, the micropatterned 3D hPSC forms a tightly controlled biochemical niche.

FIG. 9 provides a schematic representing in vitro morphogenesis in three differentiation protocols in accordance with embodiments. (i) Exposure to BMP, without neural induction, results in an amnion-like tissue containing cells from three germ layers without formation of a neural fold. (ii) Neural tube morphogenesis is observed when neural induction is followed by exposure to BMP4. (iii) Homogenous expression of forebrain markers is observed under exposure to neural induction without BMP.

FIG. 10 provides a schematic and results of the experimental timeline for three protocols detailed in FIG. 9. (B) Experimental timeline for the three protocols. Neural induction media includes N2 supplement and TGFβ inhibitor SB-431542 (SB). (C) Vertical sections of immunostained samples from the three differentiation protocols.

FIG. 11 provides a three-dimensional reconstruction of a stem cell derived ˜1 mm long neural tube and images of horizontal section through a micropatterned array of stem-cell derived neural tubes with reproducible morphology, generated in accordance with embodiments. Scale bar 500 μm.

FIG. 12A provides images of horizontal sections of 16 circular cultures from a single array exhibit stereotypic fate-patterning and morphology, generated in accordance of embodiments.

FIG. 12B provides a bar plot showing success rate of neural tissue folding morphogenesis, in 13 experiments, with total N=100 samples, generated in accordance with embodiments.

FIG. 13 provides a schematic and images depicting folding morphogenesis observed in three-dimensional cell cultures in accordance with embodiments. (a) Scheme showing experimental design and results. Induction of neural pattern formation in 3D stem cell cultures with a lumen triggers folding morphogenesis. In contrast triggering pattern formation in 2D micropatterned cultures does not trigger folding morphogenesis. (b) Vertical sections of 2D immunostained samples and (b) 3D immunostained samples. NCAD is shown in red, ECAD in cyan, and SOX10 in green. Scale bar is 50 μm.

FIG. 14 provides images of stages of neural folding in the stem cell system, generated in accordance with embodiments. Scale bars 50 μm.

FIG. 15 provides images of neural closure, generated in accordance with embodiments. Neural closure is mediated by fusion of the non-neural ectoderm layer. (i) An actin ring is observed during neural closure. Scale bars 50 μm (h) and 25 μm (i).

FIG. 16 provides a schematic and images of neural-ectoderm bilayer formation in accordance with embodiments. (a) Scheme of a neural-ectoderm bilayer formed during stem-cell fold morphogenesis. The neural-ectoderm interface is enriched with extracellular matrix, and neural crest cells. (b) Vertical and (c-k) horizontal sections through the stem cell derived neural tube. Immunostaining with mesoderm marker Brachyury (BRA) reveals no mesoderm tissue is involved in neural folding. Samples additionally immunostained with anterior neural markers PAX6 and OTX2 (d,e); epidermal markers TFAP2a and Kaertain8 (KTN8) (f,g); Neural crest markers SOX10 and PAX7(h,i); Focal adhesion marker phosphorylated focal adhesion kinase (pFAK), and extracellular matrix marker fibronectin (j,k). Scale bars 50 μm (b,c) 25 μm (d-k).

FIG. 17 provides a schematic and images of folding morphogenesis, which occurs in the absence of mesendoderm tissue, in accordance with embodiments. (A) Experimental time line to examine effect of neural induction on cell fate and folding morphogenesis. (B-D) Vertical and horizontal sections showing the neural fates (NCAD) are upregulated with longer neural induction, whereas mesendoderm fates (Brachyury) are downregulated. (E, F) A small number of Brachyury+ cells (<10) is present in the protocol used to for neural tube morphogenesis. Total number of cells in the tissue is ˜5000 cells. Scale bar is 50 μm.

FIG. 18 provides a schematic and images of neural differentiation in the absence of BMP4 in which no neural folding is observed, in accordance with embodiments. (A) Experimental timeline. Neural induction media contains TGFβ inhibitor SB-431542 (SB). (B) Bright field images showing 3D tissue growth and over 7 days. (C) At day 7 of neural induction neural markers OTX2 and PAX6 are expressed. A single lumen is maintained, as indicated by tight junction marker ZO-1. Cells are radially organized as indicated by Tubulin. No non-neural tissue is observed, and neural folding does not occur. Scale bar is 50 μm.

FIG. 19 provides a schematic, images, and bar graph of cellular differentiation in the absences of neural induction in which no neural folding is observed, in accordance with embodiments. (A) Experimental timeline for BMP4 exposure without neural induction. (B) Bright field images showing development over 3 days. At day 4, cells migrate away from the micropatterned area. (C) Horizonal and (D) vertical sections of samples at day 3. Micropattern diameter is indicated in microns. Cells close to the glass express mesendoderm markers Brachyury (BRA) and SOX17. In 450 μm patterns, SOX2 is observed at the center of the tissue. Cells at the colony edges and at the top express amniotic ectoderm marker CDX2. A single lumen is maintained and no spontaneous folding is observed. (E) qPCR analysis for differentiation markers as a function of BMP concentration. BMP results in upregulation of the WNT and NODAL pathways, upregulation of mesendoderm markers EOMES/Brachyury/SOX17, amniotic ectoderm marker GATA3, downregulation of ectoderm marker SOX2 and pluripotency marker NANOG. Scale bar 50 μm unless indicated otherwise.

FIG. 20 provides schematics of in vivo and in vitro neural folding based on neural plate size in accordance with embodiments. (a) Scheme showing changes in neural plate size and neural tube shape along the anterior posterior axis. (b) Experimental design-micropatterns are created at increasing widths to study the effect of tissue size on fold morphology.

FIG. 21 provides images and a data graph of in vitro neural folding based on neural plate size, generated in accordance with embodiments. (c) Horizonal sections show neural (NCAD, red) and ectoderm (ECAD,cyan) pattern formation at varying micropattern widths. Scale bar 100 μm. (e) Plot of neural and ectoderm tissue width as a function of micropattern size. White area indicates the region in which a single hinge is observed. Grey area indicates the region in which two hinges are observed.

FIG. 22 provides images of vertical section that reveal neural fold morphology changes as micropattern width increases, generated in accordance with embodiments. At 150 μm a single medial hinge is observed, whereas larger micropatterns result in two lateral hinges. Scale bar 50 μm.

FIG. 23 provides a data plot of the ratio of apical to basal neural area as a function of micropattern size, generated in accordance with embodiments. The apical-basal ratio is independent of micropattern size and is indicative of apical contractility.

FIG. 24 provides a schematic of neural tube morphology in control sample and following exposure to ROCK inhibitor, novobiocin, or valproic acid, in accordance with embodiments.

FIG. 25 provides images of neural folding in the presence of ROCK inhibitor, novobiocin, or valproic acid, generated in accordance with embodiments. (b) Vertical sections show neural fold defect in control sample, (c) with ROCK inhibitor Y-27632, (d) fibronectin matrix inhibitor novobiocin, and (e) NTD-associated valproic acid. (b′) High magnification of neural (red) and (b″) non-neural ectoderm (cyan) apical cell borders in control and (c′,c″) ROCK inhibited samples. Scale bars 50 μm (b-e), 5 μm (b′, b″, c′, c″).

FIG. 26 provides data graphs of neural folding in the presence of ROCK inhibitor, novobiocin, or valproic acid, generated in accordance with embodiments. (a) Analysis of neural apical curvature, (b) total neural apical area, and (c) neural/non-neural (N-E) interface in control (CTRL) and treated samples. (d) Apical F-actin and (e) Apical cell area and quantified in CTRL and ROCK inhibited samples. Data are mean+/−s.d.

FIG. 27 provides images and data plots of neural folding in the presence of ROCK inhibitor Y-27632, generated in accordance with embodiments. (a) Horizonal sections show Shroom3 and F-actin localization to neural apical surface (N. Api.). (b) F-Actin and (c) Shroom3 fluorescence intensity across the apical-basal axis in CTRL (solid black line) and ROCK inhibitor (grey dashed line) samples. Scale bars 10 μm.

FIG. 28 provides a schematic of cardiac differentiation in accordance with embodiments.

FIG. 29 provides images of cardiac organoid formation via the differentiation protocol provided in FIG. 28, generated in accordance with embodiments.

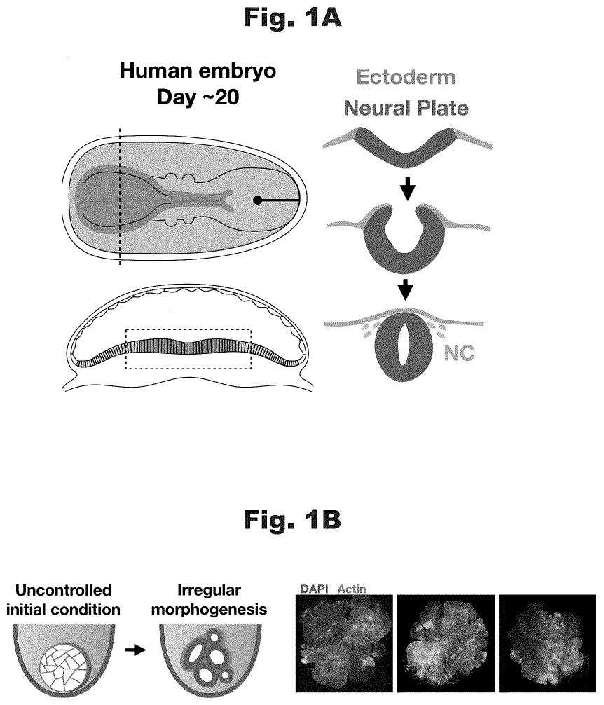

### DETAILED DESCRIPTION

Turning now to the drawings and data, systems and methods to form a cellular lumen are provided. In many embodiments a cellular lumen is a sheet of interconnected epithelial cells formed into a dome-like or tubular structure with a lumen therein. In various embodiments, a cellular lumen is formed via in vitro culturing of animal cells, utilizing cellular matrices and micropatterning. Multiple embodiments are also directed to systems and methods for cellular differentiation utilizing a three-dimensional lumen. Numerous tissue types form lumens in their natural differentiation process in embryonic and adult animal tissue development, including (but not limited to) neurogenesis, vasculogenesis, angiogenesis, cardiogenesis, nephrogenesis, gastrulation, and lung formation. Accordingly, various embodiments are directed towards in vitro tissue differentiation of various cell types utilizing lumen formation, including (but not limited to) differentiation of cells involved in neurogenesis, vasculogenesis, angiogenesis, cardiogenesis, nephrogenesis, gastrulation, and lung formation. Further, several embodiments are directed towards utilizing systems and methods of lumen formation and/or cellular differentiation in a variety of applications, including (but not limited to) disease modeling, drug screening, and personalized medicine.

### Lumen Formation

A number of embodiments are directed to forming a cellular lumen in vitro utilizing cellular culture techniques. A lumen, as understood in the biological art, is a cavity or channel within a tubular biologic structure. Typically, the luminal structure is a closed sheet of interconnect cells, especially epithelial cells. In various embodiments, lumen formation is performed by exposing cells to a particular pattern of proteinaceous matrix on a substrate; then cellular media with additional suspended matrix is added such that the cells form a lumen.

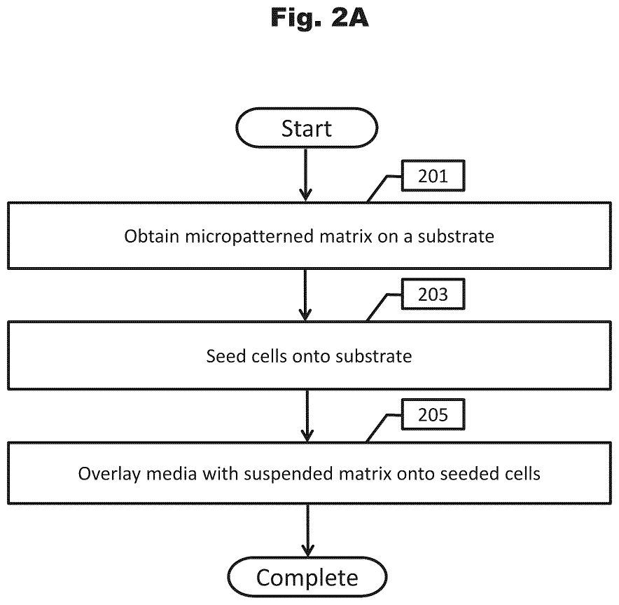

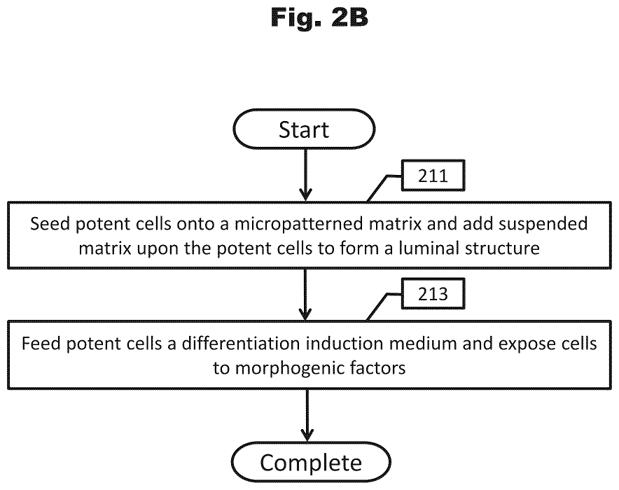

Provided in FIG. 2A is an embodiment of a method to formulate a cellular luminal structure having a lumen. The method can begin by obtaining 201 a micropatterned matrix on a substrate. A micropatterned matrix is a cell-supportive matrix provided in a particular shape having dimensions at a micrometer scale. Matrices are typically organic substances comprising proteinaceous components derived from an animal source, but any appropriate matrix may be utilized, including (but not limited to) organic matrices, animal-derived matrices, proteinaceous matrices, non-animal-derived matrices, inorganic matrices, and/or synthetic matrices. Common matrices utilized include collagen, laminin, fibronectin, elastin, alginate, poly-lysine, poly-arginine, polysaccharide, Matrigel (Corning Life Sciences, Corning, NY), and Geltrex (ThermoFisher Scientific, Waltham, MA). In some instances, matrices can be combined and/or sequentially coated onto a substrate (e.g., poly-lysine and laminin; or collagen and fibronectin). The matrix utilized will likely depend on the cell type utilized to form a lumen, as each cell type has different attachment and extracellular signaling requirements to maintain vitality and morphology. For instance, matrices often utilized for embryonic stem cells include (but are not limited to) laminin, Matrigel, and Geltrex. Further, any appropriate substrate may be utilized. Various substrates for cell culture include glass, polystyrene, polytetrafluoroethylene, thermamox, polyvinylchloride, polycarbonate, agar, agarose, sephadex, polyacrylamide, and palladium. In some instances, a substrate is treated to promote adhesion. For example, polystyrene can be treated with gamma radiation and glass can be treated with plasma.

A micropatterned matrix can be obtained by fabricating the matrix shape. In some instances, lithographic technique is utilized. For instance, in some embodiments, an appropriate cell-culture substrate is stamped with a material (e.g., polydimethylsiloxane) in the desired micropattern shape. The unstamped portion of the cell-culture substrate is passivated or blocked with an appropriate material to prevent matrix and/or cell binding. In some instances, poly(ethylene glycol) (PEG) is utilized. In some instances, PEG is co-polymerized with a material (e.g., poly-lysine) to promote its interaction with the substrate surface. After passivating or blocking the surface of the unstamped area, the stamps are removed and then the substrate is coated with a matrix. The matrix will specifically interact with the micropatterned regions (i.e., the stamped regions) and will not interact with the unstamped regions as these regions have been passivated or blocked. In various embodiments, a substrate is coated with matrix on one or more micropattern regions. In some embodiments, a plurality of micropatterned matrices are formed on a substrate surface. In some embodiments, each micropatterned matrix of a plurality of micropatterned matrices have the same micropattern. In some embodiments, a plurality of micropatterned matrices are formed on a substrate surface such that the plurality of micropatterned matrices comprise at least two different micropatterns (e.g., micropatterns with different shape or different dimensions).

Any micropatterned shape may be utilized, including circular, ovular, triangular, rectangular, or any other two-dimensional shape. To form a dome-shaped luminal structure, the micropattern should be circular or equilaterally shaped. Accordingly, in various embodiments to form a dome-shaped luminal structure, a circular or equilaterally shaped micropattern has a midline that is between about 100 microns and about 1000 microns. In various embodiments, the midline of a micropattern is about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. In the context of micropattern midline, the term “about” refers to width plus or minus 50 μm. For example, a midline of about 500 μm refers to a midline between 450 μm and 550 μm. Furthermore, the edges of a micropattern can be straight or curved.

To form a semi-tubular luminal structure, the micropattern should have a shape with a width and length. The width of the micropattern should match the desired cross section of the semi-tubular lumen to be formed. Further, the length of the micropatterned shape should match the desired length of the semi-tubular lumen. Accordingly, in various embodiments to form a semi-tubular luminal structure, a micropattern has a width that is between about 100 microns and about 1000 microns and a length longer than the width. In some embodiments, the width of a micropattern is varied along the length, which will result in a semi-tubular lumen having a cross section that varies in accordance with the micropattern width. In various embodiments, the width of a micropattern is about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, and/or about 1000 μm. In the context of micropattern width, the term “about” refers to width plus or minus 50 μm. For example, a width of about 500 μm refers to a width between 450 μm and 550 μm. Furthermore, the edges of a micropattern can be straight or curved.

As shown in the exemplary method provided in FIG. 2A, biological cells are seeded 203 onto the substrate, resulting in a micropatterned two-dimensional layer of cells upon the micropatterned region(s). In some embodiments, the cells express adhesion molecule proteins (such as cadherins, integrins, and selectins) that result an interconnected cell layer. Notably, the layer of cells can be a single monolayer (including pseudostratified), or in layers of two or more cells (e.g., stratified and transitional multi-cell layers). In some embodiments, the cells are epithelial cells, which are cells that formulate an epithelium. Epithelial cells include squamous, cuboidal, and columnar cell types and can originate from a variety of animal organs. Some epithelial cells are transitory during development, such as (for example) the trophoectoderm and cells of the neural tube. Furthermore, numerous cells cultured in vitro form an epithelium, including (but not limited to) embryonic stem cells, induced pluripotent stem cells, neural stem cells, primary epithelial cells, intestinal epithelial cells, endothelial cells, primary endothelial cells, cardiac endothelial cells, pulmonary epithelial cells, pancreatic epithelial cells, gastric epithelial cells, renal epithelial cells, liver epithelial cells, neuroepithelial cells, and skin epithelial cells.

In some embodiments, cells used for seeding are previously treated to form a single-cell suspension. Utilization of a single-cell suspension can prevent cell clumping onto the micropattern and can promote smooth two-dimension epithelial layers. It is to be understood that a single-cell suspension is a suspension with greater than 50% of cells detached from any other cell. In various embodiments, the single-cell suspension is a cell suspension with at least 50% single cells, at least 60% single cells, at least 70% single cells, at least 80% single cells, at least 90% single cells, and/or at least 95% single cells. The appropriate seeding density can vary and depends on the cell type and the area of micropatterned regions. After seeding, the cell suspension can be washed off to remove any excess cells that did not attach to the micropatterned regions. Further, the seeded cells should be overlaid in an appropriate medium with factors to maintain their vitality and any other appropriate features (e.g., potency).

FIG. 2A further displays that media with suspended matrix is overlaid 205 onto the seeded cells, which allows the layer of epithelial cells within the micropattern to form a luminal structure with a luminal floor and luminal wall comprising cells. The luminal floor is the layer of cells upon the micropatterned matrix and the luminal wall extends from the edges of the luminal floor and is interconnected to form a closed dome-like or semi-tubular structure. To allow for closed lumen formation, in some embodiments, seeded cells with suspended matrix in the media is left unperturbed for a time period of at least 8 hours. In various embodiments, the seeded cells with suspended matrix in the media is left unperturbed for at least 8 hours, at least 12 hours, at least 16 hours, at least 20 hours, at least 24 hours, at least 36 hours, at least 48 hours, at least 60 hours, at least 72 hours, and/or until substantially semi-circular or semi-ovular. The length of lumen formation depends on the cell type and the size of the micropattern. Micropatterns with greater widths will typically require greater time for lumen formation than micropatterns with less width. The concentration of suspended matrix in media will depend on the type of matrix utilized and the cell type. In many embodiments, at least 1% of Matrigel (v/v) is utilized for a number of cell types. For example, it has been found that pluripotent stem cells in a media containing 1-8% Matrigel (v/v) promoted formation of a lumen, and specifically 2-4% Matrigel (v/v) assisted in neural tube formation and differentiation from pluripotent stem cells. It has further been found that up to 100% Matrigel (v/v) can be utilized, but higher concentrations of Matrigel may yield difficulty in maintaining and/or controlling potency and/or cell differentiation lineage. Various lumen shapes can be formed in accordance with the micropatterned shape, as described previously.

Numerous embodiments are also directed towards systems for cellular luminal structure formation. Accordingly, various embodiments of systems may include one or more essential components for cellular luminal structure formation. In some embodiments, a system for cellular luminal structure formation includes a micropatterned matrix layered upon a substrate, biological cells in contact with the micropatterned matrix, and media containing suspended matrix in contact with the biological cells.

### Cellular Differentiation

Several embodiments are directed to differentiating potent cells in vitro utilizing luminal structure formation. Cellular differentiation is the process of maturing a more potent cell into a less potent cell. Any differentiation protocol can be utilized with lumen formation, especially protocols for cell types that utilize a lumen during in vivo differentiation. Numerous cell types that utilize a lumen during in vivo development include (but are not limited to) trophectoderm cells, ectoderm cells, neural tube cells, nephrons, and endoderm cells. Accordingly, luminal structure formation can be utilized in various in vitro cell culture differentiation techniques that mimic in vivo cellular lumen formation during development.

Provided in FIG. 2B is a method to differentiate cells in vitro utilizing luminal structure formation. The method can begin by seeding 211 potent cells onto a micropatterned matrix and adding suspended matrix upon the potent cells to form a luminal structure. A potent cell is any cell with the ability of differentiating into a more mature cell type. Potent cells include (but are not limited to) totipotent cells, pluripotent cells, multipotent cells, unipotent cells, mammalian stem cells, embryonic stem cells, induced pluripotent stem cells, adult stem cells, epithelial stem cells, endothelial stem cells, neural stem cells, renal stem cells, angioblasts, cardiac stem cells, intestinal stem cells, pancreatic stem cells, lung stem cells, vascular stem cells, and skin stem cells.

A micropatterned matrix, as stated previously, is a cell-supportive matrix provided in a particular shape having dimensions at a micrometer scale. Any appropriate matrix may be utilized, including (but not limited to) organic matrices, animal-derived matrices, proteinaceous matrices, non-animal-derived matrices, inorganic matrices, and/or synthetic matrices. Common matrices utilized include bovine serum, collagen, laminin, fibronectin, elastin, alginate, poly-lysine, poly-arginine, polysaccharide, Matrigel (Corning Life Sciences, Corning, NY), and Geltrex (ThermoFisher Scientific, Waltham, MA). In some instances, matrices can be combined and/or sequentially coated onto a substrate (e.g., poly-lysine and laminin; or collagen and fibronectin). The matrix utilized will likely depend on the potent cell type utilized to form a lumen, as each cell type has different extracellular signaling requirements to maintain vitality and morphology. For instance, matrices often utilized for embryonic stem cells include (but are not limited to) laminin, Matrigel, and Geltrex.

Any micropatterned matrix shape may be utilized, including circular, ovular, triangular, rectangular, or any other two-dimensional shape. In various embodiments to form a dome-shaped luminal structure, a circular or equilaterally shaped micropattern has a midline that is between about 100 microns and about 1000 microns. In various embodiments, the midline of a micropattern is about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. In the context of micropattern midline, the term “about” refers to width plus or minus 50 μm. For example, a midline of about 500 μm refers to a midline between 450 μm and 550 μm. Furthermore, the edges of a micropattern can be straight or curved.

In some embodiments, a semi-tubular luminal structure is desired for cellular differentiation. Accordingly, to form a semi-tubular luminal structure, the micropattern should have a shape with a width and length. The width of the micropattern should match the desired cross section of the semi-tubular lumen to be formed. Further, the length of the micropatterned shape should match the desired length of the semi-tubular lumen. In various embodiments to form a semi-tubular lumen, a micropattern has a width that is between about 100 microns and about 1000 microns and a length longer than the width. In some embodiments, the width of a micropattern is varied along the length, which will result in a semi-tubular lumen having a cross section that varies in accordance with the micropattern width. In various embodiments, the width of a micropattern is about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, and/or about 1000 μm. In the context of micropattern width, the term “about” refers to width plus or minus 50 μm. For example, a width of about 500 μm refers to a width between 450 μm and 550 μm. Furthermore, the edges of a micropattern can be straight or curved.

In some embodiments, potent cells used for seeding are previously treated to form a single-cell suspension. Utilization of a single-cell suspension can prevent cell clumping onto the micropattern and can promote smooth two-dimension epithelial layers. It is to be understood that a single-cell suspension is a suspension with greater than 50% of cells detached from any other cell. In various embodiments, the single-cell suspension is a cell suspension with at least 50% single cells, at least 60% single cells, at least 70% single cells, at least 80% single cells, at least 90% single cells, and/or at least 95% single cells. The appropriate seeding density can vary and depends on the cell type and the area of micropatterned regions. After seeding, the cell suspension can be washed off to remove any excess cells that did not attach to the micropatterned regions. The seeded cells should be overlaid in an appropriate medium with factors to maintain their vitality.

Suspended matrix is added to the medium to induce luminal structure formation. In some embodiments, suspended matrix is added to a medium that promotes maintenance of the cells' current potency. In some embodiments, suspended matrix is added to a medium that promotes cellular differentiation. To allow for closed lumen formation, in some embodiments, seeded cells with suspended matrix in the media is left unperturbed for a time period of at least 8 hours. In various embodiments, the seeded cells with suspended matrix in the media is left unperturbed for at least 8 hours, at least 12 hours, at least 16 hours, at least 20 hours, at least 24 hours, at least 36 hours, at least 48 hours, at least 60 hours, at least 72 hours, and/or until substantially semi-circular or semi-ovular. The concentration of suspended matrix in media will depend on the type of matrix utilized and the cell type. For example, it has been found that pluripotent stem cells in a media containing 2-8% Matrigel (v/v) promoted formation of a luminal structure.

As shown in FIG. 2B, potent cells are fed 213 a differentiation induction medium and exposed to morphogenic factors. A differentiation induction medium is a medium that includes and/or excludes factors that promote cellular differentiation. Morphogenic factors compounds that promote cellular differentiation and include (but are not limited to) small molecules, peptides, proteins, lipid moieties, and nucleic acid components. In some embodiments, the differentiation induction medium is fed to the cells during the seeding process. In some embodiments, the differentiation induction medium is fed to the cells after the seeding process. In some embodiments, the differentiation induction medium is fed to the cells after lumen formation.

The factors and components to be included within a differentiation induction medium depend on the cell type desired. Typically, components and morphogenic factors utilized in a differentiation induction medium mimic the in vivo environment where differentiation occurs naturally. For instance, in some protocols to differentiate embryonic stem cells into neural cells, the embryonic stem cell medium promoting the maintenance of pluripotency is replaced with a medium containing morphogenic factors of TGFβ-inhibitor, BMP4, and/or FGF2. A number of differentiation induction media can be utilized, including (but not limited to) neural induction media, renal induction media, angiogenesis induction media, cardiac induction media, intestinal induction media, pancreatic induction media, lung induction media, vascular induction media, and skin induction media. Numerous induction media have been described in the literature and/or sold commercially. For instance, see “SnapShot: directed differentiation of pluripotent stem cells” for induction media and factors for differentiation of pluripotent stem cells into various cellular lineages (L. A. Williams, B. N Davis-Dusenbery, and K. C. Eggan; 2012; 149(5): 1174-1174.e1, the disclosure of which is incorporated herein by reference).

The exposure timeline and concentration of morphogenic factors depends on the factor and the differentiation protocol to yield the desired cell type. In some instances, one or more morphogenic factors are exposed to cells immediately when the induction media is initially fed to the cells. In some instances, one or more morphogenic factors are added at a timepoint after the induction media is initially fed to the cells. In some instances, the concentration of a morphogenic factor is varied over time, as dependent on the differentiation protocol. Several morphogenic factors have been described in the literature and/or sold commercially to promote differentiation of various cell types. For instance, morphogenic factors of TGFβ-inhibitor, BMP4, and/or FGF2 can be utilized to promote neural differentiation.

When utilizing cellular luminal structure formation in conjunction with differentiation, spatial signaling and patterning can be performed. For instance, it has been found that lumen formation utilized in conjunction with neural induction media and morphogenic factors resulted in the formation of a neural-tube like-structure with dorsal-ventral patterning, mimicking the three-dimensional shape and patterning of neural tube formation as seen in vivo. In some embodiments, morphogenic factors can be concentrated towards various sections along the lumen, which may help promote particular differentiation patterning.

Numerous embodiments are also directed towards systems for cellular differentiation with luminal structure formation. Accordingly, various embodiments of systems may include one or more essential components for cellular differentiation and luminal structure formation. In some embodiments, a system for cellular differentiation with luminal structure formation includes a micropatterned matrix layered upon a substrate, biological cells in contact with the micropatterned matrix, media containing suspended matrix in contact with the biological cells, a differentiation induction media, and morphogenic factors.

### Applications of Cellular Lumen Formation

Various embodiments are also directed towards utilizing cellular lumens and/or cellular differentiation in an application. In some embodiments, a method and/or a system for cellular lumens and/or cellular differentiation is utilized within a disease modeling application. In some embodiments, a method and/or a system for cellular lumens and/or cellular differentiation is utilized within a personalized medicine application.

In vitro disease modeling is the recapitulation of a disease utilizing cellular culture. Typically, cells utilized for disease modeling have a genetic modification and/or are perturbed in a manner that gives rise to the disease to be modeled. In vitro disease modeling is especially useful for studying diseases on a cellular and molecular level. In some embodiments, a disease is modeled in combination with a method and/or system for cellular lumen formation. In some embodiments, a disease is modeled in combination with a method and/or a system for cellular lumen formation with cellular differentiation. For instance, neural tube defects (such as, e.g., spina bifida, anencephaly, craniorachischisis) can be modeled utilizing a method and/or system for cellular lumen formation with cellular differentiation (see Exemplary Embodiments for further details). Additionally, medical conditions related to neural crest differentiation and migration can be modeled, including, but not limited to, Waardenburg's syndrome, craniofacial birth defects, Treacher Collins syndrome, Hirschsprung disease, and neuroblastoma.

In vitro drug screening is the experimental procedure of a testing various compounds utilizing cellular culture. Typically, in vitro drug screening is performed on a cellular disease model that recapitulates an aspect of the disease that is to be treated. In vitro drug screening is especially useful for identifying drug candidates, especially novel drug candidates. In some embodiments, drug screening is performed in combination with a method and/or system for cellular lumen formation. In some embodiments, drug screening is performed in combination with a method and/or a system for cellular lumen formation with cellular differentiation. In some embodiments, drug screening is performed to identify drug candidates that provide an ameliorative effect. In some embodiments, drug screening is performed to identify potential unintended consequences (e.g., undesirable side effects). For example, a drug compound can be tested to determine an effect during in utero development (e.g, effect on neural tube formation) to determine whether the drug compound would have unintended effects on a fetus if taken during pregnancy.

Personalized diagnostics and medicine utilize an in vitro experimental procedure of a screening various drug compounds on a personalized cellular culture. Typically, a personalized cellular culture model is created that recapitulates the individual's cells. To create a personalized cellular culture model, cells of interest are extracted from an individual and cultured in vitro. In some instances, the extracted and cultured cells are induced into a more potent state. For example, an individual's fibroblasts (i.e., skin cells) can be extracted and cultured and then induced into a pluripotent stem cell. In some instances, extracted cells and/or induced potent stem cells are manipulated and/or differentiated into the cell type that is to be utilized for diagnostic assessment and/or drug compound screening. In some embodiments, diagnostic assessment and/or personalized drug screening is performed in combination with a method and/or system for cellular lumen formation. In some embodiments, personalized diagnostic assessment and/or drug screening is performed in combination with a method and/or a system for cellular lumen formation with cellular differentiation.

Numerous embodiments are directed to experimental protocols to identify a gene and/or a mutation involved with or have an effect on lumen formation. Accordingly, in some embodiments, lumen formation protocols in conjunction with gene expression and/or gene sequence alteration protocols are utilized to identify genes that have an effect on lumen formation. Gene expression alteration protocols may involve gene expression enhancement, gene expression depression, and/or gene expression abatement. Such protocols may include (but are not limited to) overexpression assays, RNAi assays, shRNA assays, promoter or repressor manipulation, and/or chromatin altering assays. Gene sequence altering protocols may involve altering genetic code to yield a substitution, missense mutation, a nonsense mutation, a frameshift mutation, an insertion, a deletion, chromosomal alteration, duplication, inversion, and/or any combination thereof. Genetic code alterations can occur within protein coding sequence or outside protein coding region, such as a mutation within a gene expression modifier (e.g., promoter, enhancer, repressor, and/or chromatin modifier). In some embodiments, an identified gene and/or mutation involved in lumen formation is utilized in a molecular diagnostic protocol. For instance, a subject may be screened for a particular molecular gene and/or a mutation affecting lumen formation as part of a diagnostic protocol.

### Exemplary Embodiments

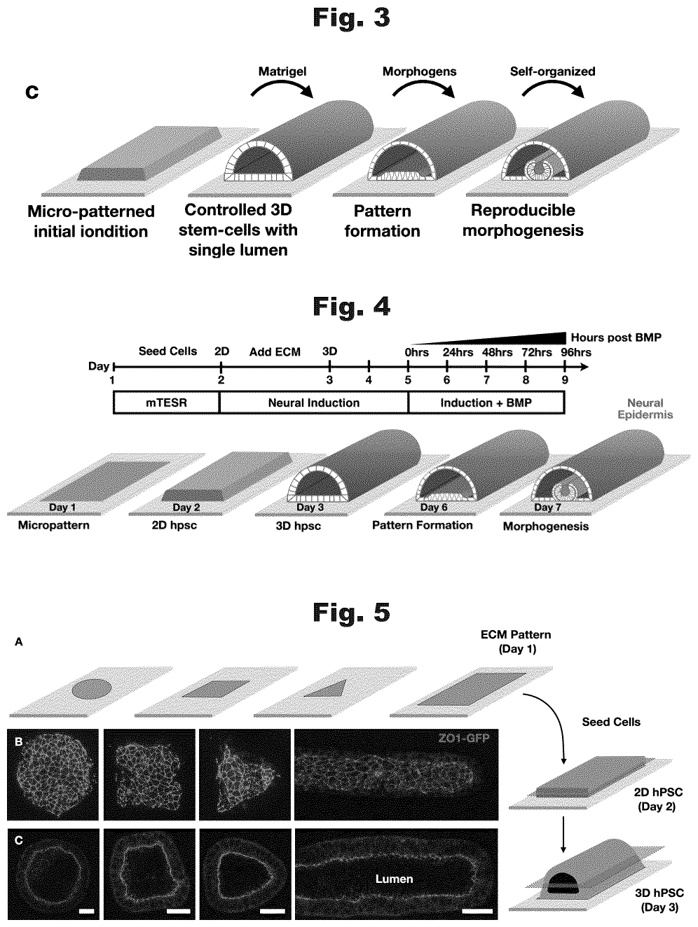

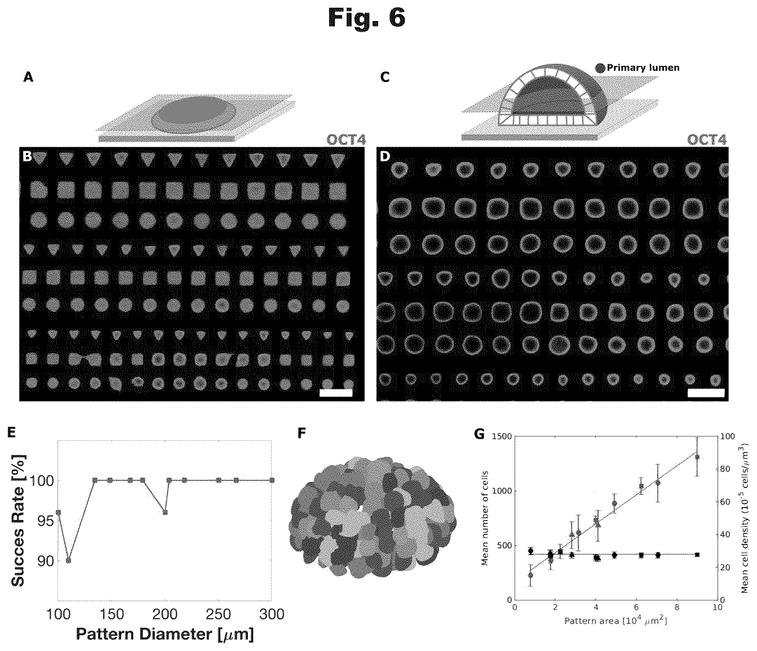

The embodiments of the disclosure will be better understood with the several examples provided within. Here, a new experimental system is described that allows faithful study of the dynamics of organ morphogenesis using human stem-cells (FIG. 3). The system is reproducible, scalable, and compatible with live imaging and genetic manipulations. The experimental design is inspired by the embryonic development of organs. Organs develop from a primary embryonic tissue, which is precisely controlled in all physical aspects including cell number, shape and size. In many cases this primordium is a two-dimensional sheet of polarized cells (epithelium) in contact with a lumen, which forms an isolated biochemical niche. This provides a well-defined physical and biochemical starting point for cell-fate patterning in the 2D epithelium that subsequently triggers 3D organ shape formation. It was thus hypothesized that controlling the initial physical conditions in which organoids develop, and realizing a 3D epithelial culture with a single lumen could reproducibly recapitulate human organ morphogenesis.

### Embryonic Cell Lumen Formation

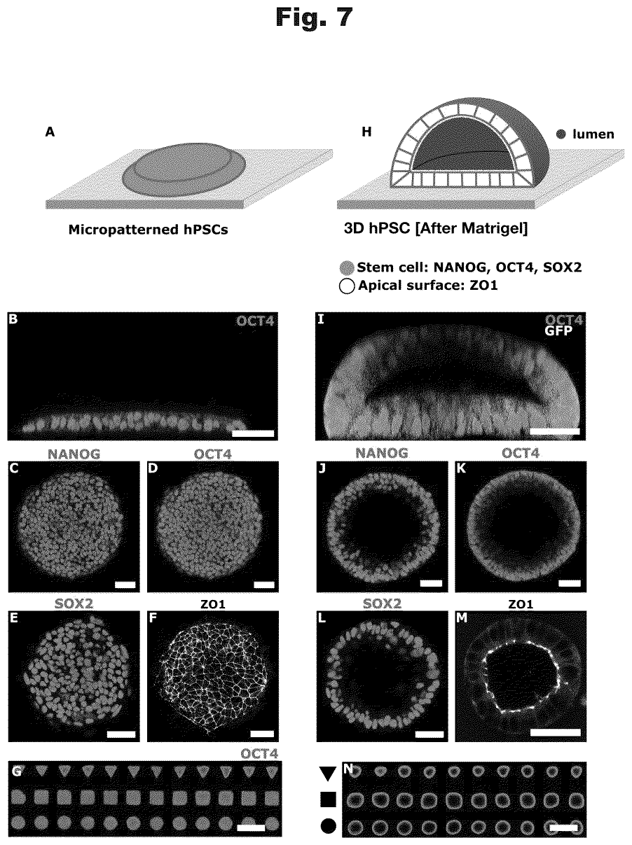

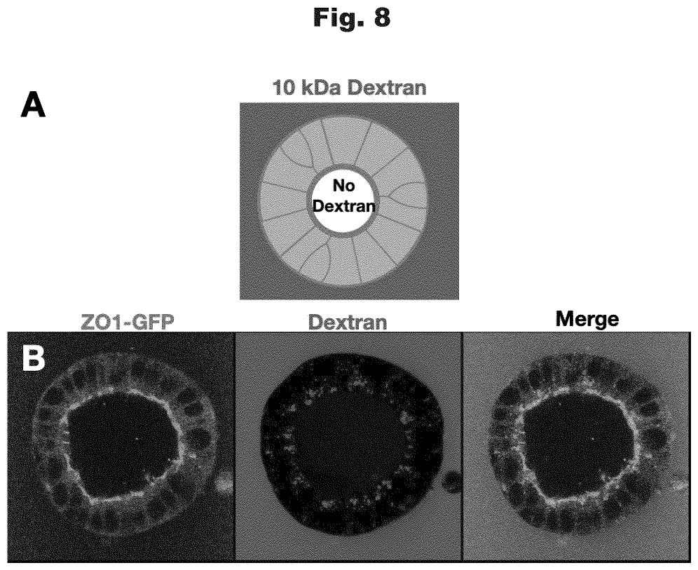

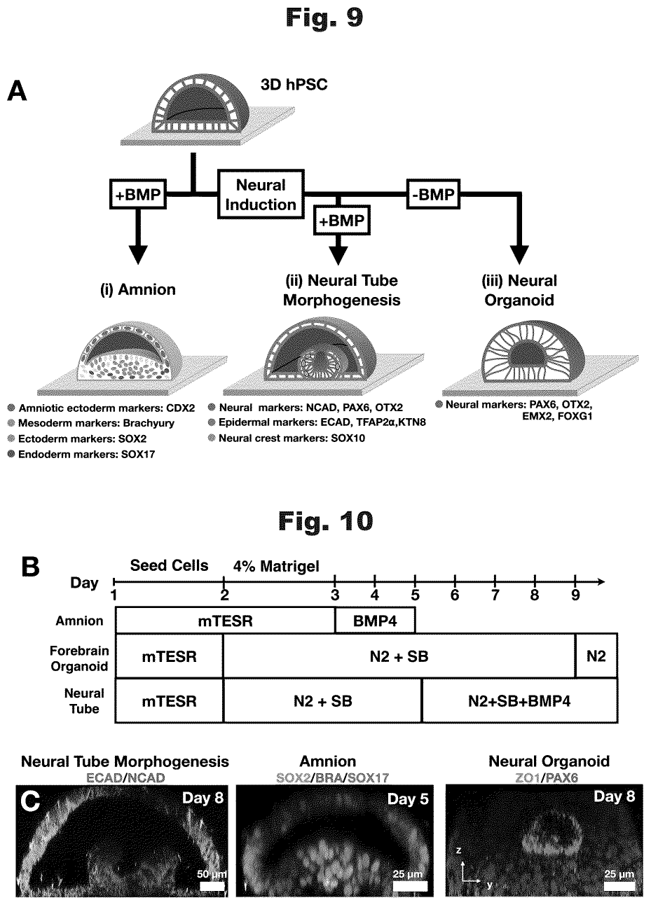

Traditionally, cultured stem cells grow in colonies that are difficult to reproduce in size, shape and differentiation potential. Here, the reproducibility challenge of stem-cell cultures was addressed by applying surface micropatterning of matrix on the substrate. The initial size, shape and cell number of 2D stem-cell cultures are precisely controlled (FIG. 4). Suspended Matrigel was then applied to trigger a robust transition from the constrained 2D culture into a 3D pluripotent epithelium that wraps around a single large lumen. The 3D stem-cell culture shape and size are controlled by the micropattern geometry and forms at >95% success rate (FIGS. 5 & 6). Importantly, the 3D tissue maintains pluripotency, and can thus give rise to many cell types of the human body (FIG. 7). Moreover, the lumen is physically and chemically isolated from the environment, thus mirroring the in vivo situation (FIG. 8). As in embryonic development, the self-organization of stem cells can be guided by exposure to morphogens. In the examples described herein, neural tube formation was performed by applying morphogens involved in early neurodevelopment. The capacity of the system to generate a human amnion and forebrain organoids is demonstrated, thus establishing a broadly applicable system for studying human organ morphogenesis (FIGS. 9 & 10).

### Neural Tube Formation

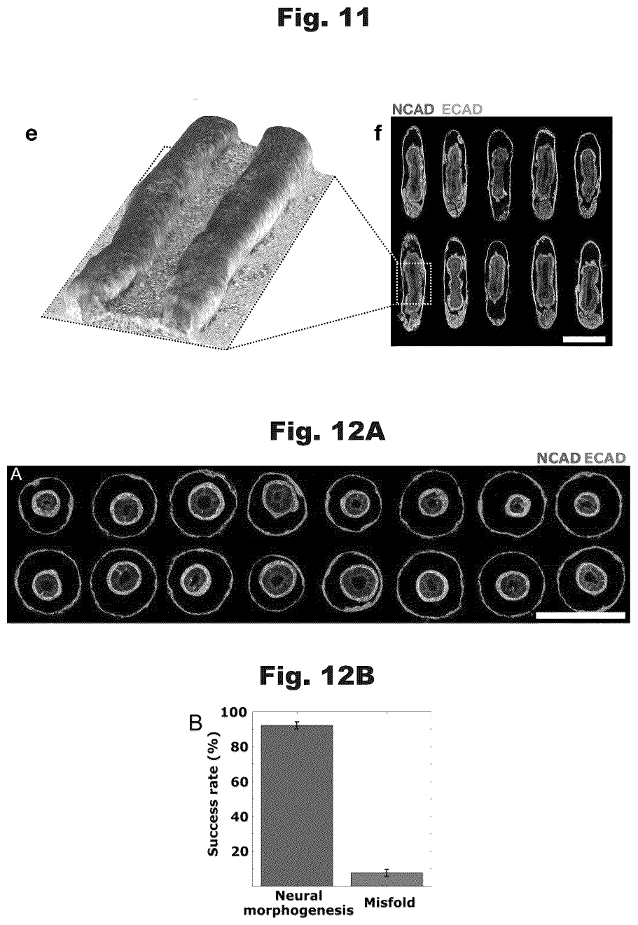

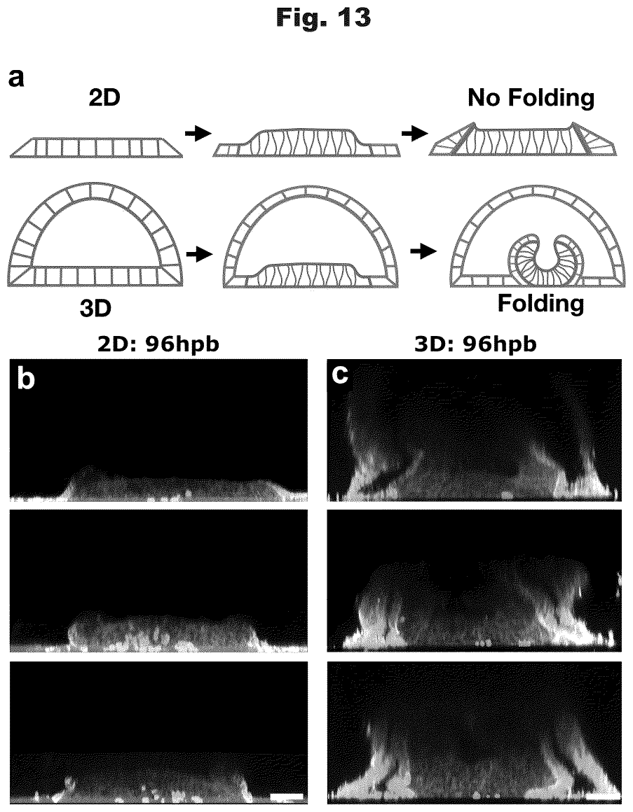

To drive neural tube morphogenesis, the 3D stem cell culture was exposed to a combination of morphogens involved in early neural development. First a neural induction media containing TGFβ inhibitor (SB-431542) was applied, followed by exposure to bone morphogenetic protein 4 (BMP4) (FIGS. 3&4). In response to the morphogens, the system exhibits self-organized folding morphogenesis which takes place over three days. The in vitro folding period is similar to neural folding in human embryos, which takes place over four days between the appearance of the neural plate and the first fusion of the neural folds. The result is a tube-shaped neural tissue covered with surface ectoderm, which recapitulates multiple anatomical features of the embryonic neural tube (FIG. 11). The process is highly reproducible and occurs with over 90% success rate in multiple cell lines (FIGS. 11, 12A & 12B). Notably, fold morphogenesis is not observed in 2D cultures, and requires a 3D tissue surrounding a lumen as an initial condition (FIG. 13).

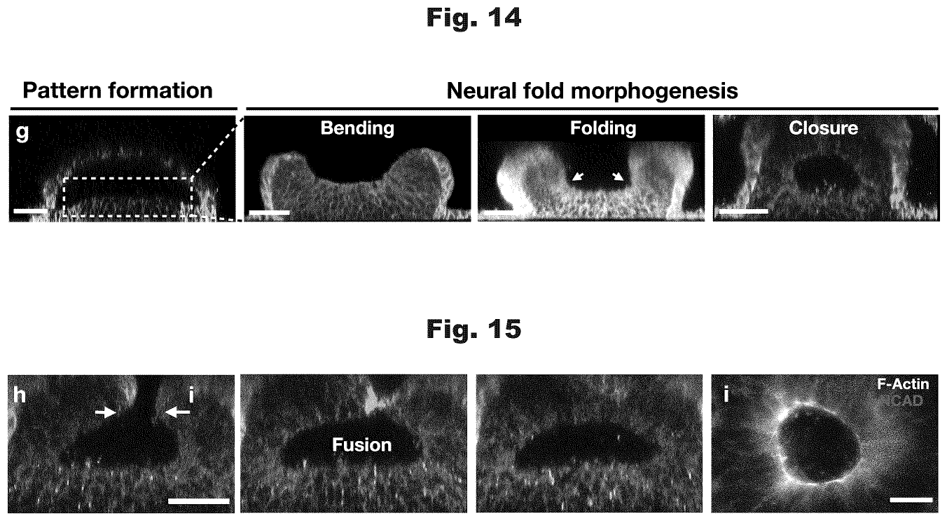

Remarkably, the in vitro neural fold morphogenesis follows the sequence of in vivo neurulation: neural plate formation and thickening, bending, folding, and closure (FIG. 14). The columnar neural epithelia dimensions, 215±15 μm width×70±5 μm thickness, are comparable to human neural plate in Carnegie stage 8. Furthermore, live imaging reveals that neural cells undergo interkinetic nuclear motion typical of neuroepithelia. Neural bending is concentrated at two focal points, which are formed at the intersection between the uprising neural folds and the glass adhered tissue. These are reminiscent of lateral hinge points which form during neural tube development in vivo. Finally, the neural closure occurs via a zippering motion in which the non-neural ectoderm makes the first contact (FIG. 15), and an actin ring is observed at the closing edge (FIG. 15), as during in vivo neural closure.

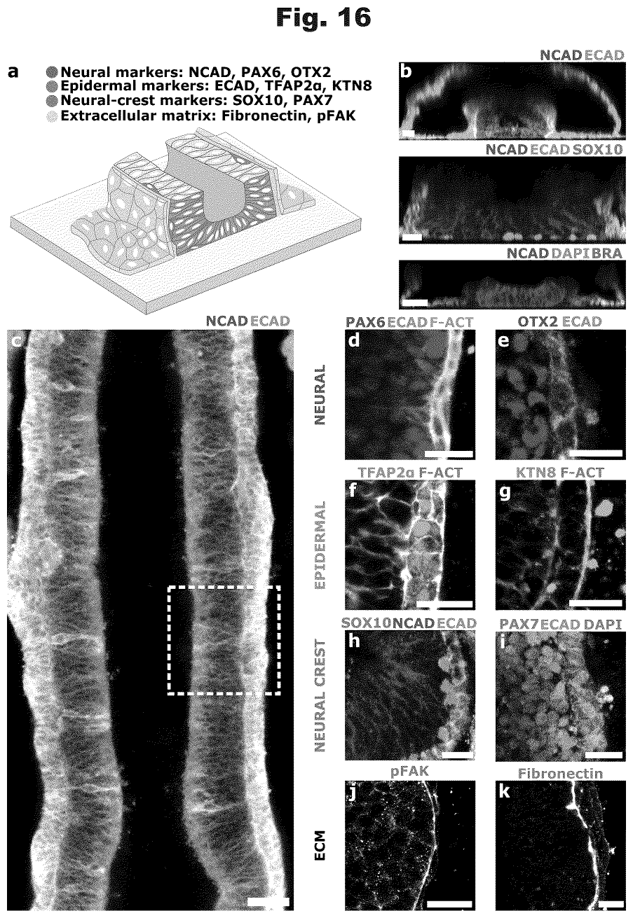

Another hallmark of neural tube development is the formation of a tissue bilayer composed of a neural layer and surface ectoderm (FIG. 16). An immunostaining study reveals that the stem cell system indeed forms a bilayer of anterior neural tissue (NCAD, PAX6, OTX2, FIG. 16), and non-neural surface ectoderm (ECAD, KTN8, TFAP2a, FIG. 16). A population of neural crest cells was observed at the neural-ectoderm interface, which coincides with their place of origin in vivo (SOX10, PAX7, FIG. 16). Interestingly, the neural-ectoderm interface is enriched in fibronectin and focal adhesions suggesting that the bi-layer is formed by basal adhesion of the two cell types (FIG. 16). Fibronectin is observed exclusively at the neural-ectoderm interface, whereas the ectoderm-glass interface is enriched with collagen. Similar ECM composition has also been observed in vivo, where the dorsal neural-ectoderm interface is enriched with fibronectin, whereas the ectoderm-mesoderm interface is enriched with collagen. Overall, these findings suggest that the in vitro model system recapitulates key aspects of in vivo neurulation, in terms of morphology and timing, cell fate patterns, extracellular matrix, and cellular behaviors.

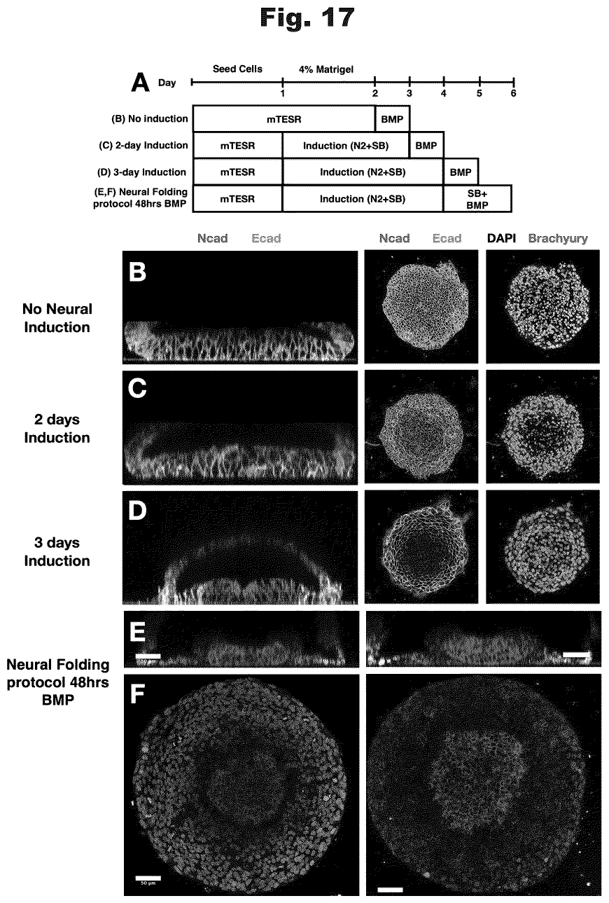

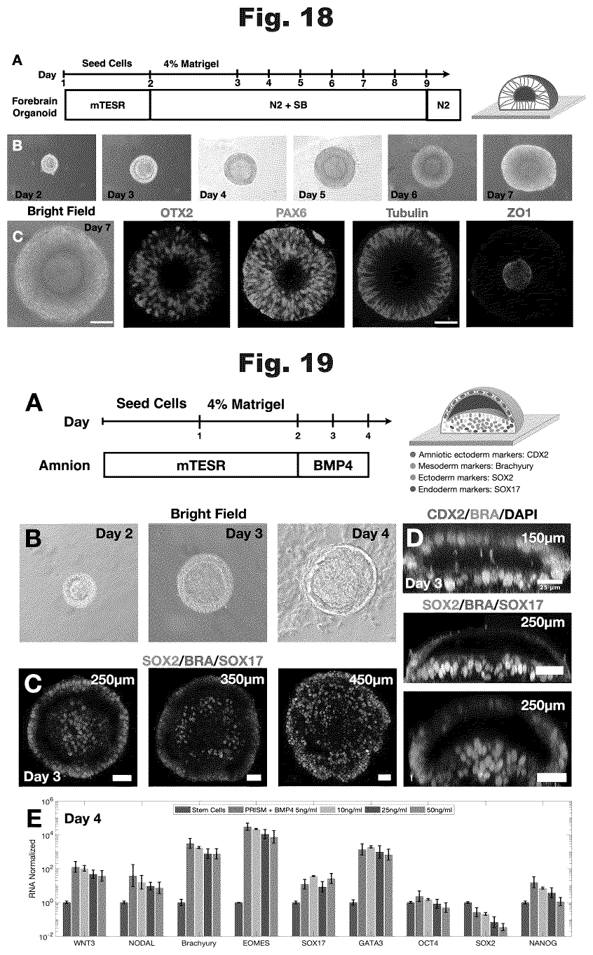

The system was next applied to study which tissues contribute to neural folding. This question has been studied for over a century in animal models, and multiple hypothesis have been developed regarding the mechanical role of the neural tissue, neighboring surface ectoderm and underlying mesoderm. However, the limited accessibility in utero led to inconclusive and often contradictive results. The in vitro system allows to systemically control tissue composition through the choice of small molecules in the culture media (FIGS. 9 & 10). This system allows discernment of the tissues which are necessary and sufficient for neural tube morphogens. Immunostaining with mesoderm marker Brachyury shows that there is no mesoderm tissue present during folding morphogenesis (FIG. 16). Mesoderm fates are suppressed by TGFβ inhibition, and only appear if the period of TGFβ inhibition is decreased (FIG. 17). Thus, the data suggest that mesoderm is not required for folding morphogenesis in vitro. It was next examined whether the non-neural ectoderm is required for folding. For this purpose, the differentiation protocol utilized included prolonged neural induction without BMP4. This results in a homogenous neural tissue that lacks epidermal markers, and does not exhibit folding morphogenesis (FIG. 18). It was observed that addition of BMP4 without neural induction lead to mesoderm and ectoderm cell fates, but lack neural tissue and does not exhibit folding (FIG. 19). It was thus concluded that epidermal and neural tissues are necessary and sufficient to drive folding morphogenesis.

### Modulation of Neural Plate Size

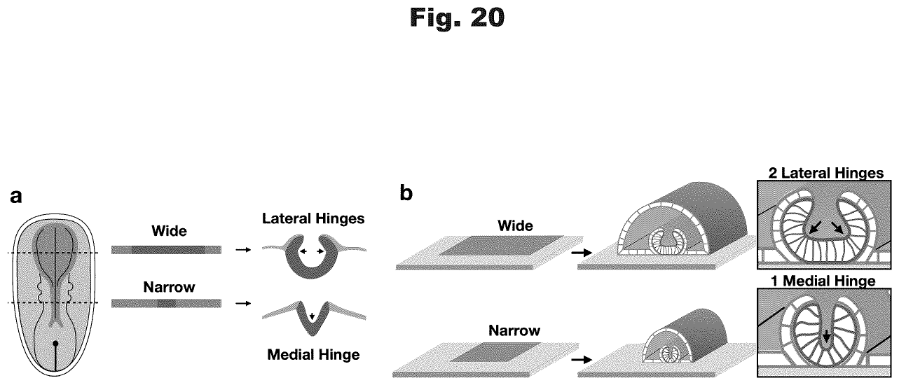

The neural plate width varies along the anterior-posterior (AP) axis, from ˜500 μm on anterior brain region down to 100-200 μm on the posterior end (FIG. 20). Neural fold morphology also varies along the anterior-posterior axis, exhibiting a broad fold with two lateral hinges at the anterior end, and narrow fold with a single medial hinge at the posterior end. The current paradigm is that neural tube morphology is controlled by cell behaviors driven by a gradient of signaling molecules along the AP axis. However, to which extent the neural plate size directly controls shape remains unclear.

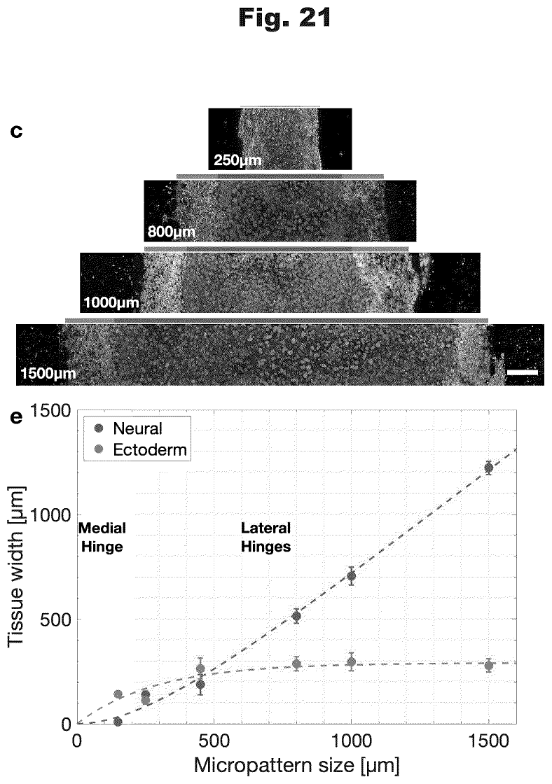

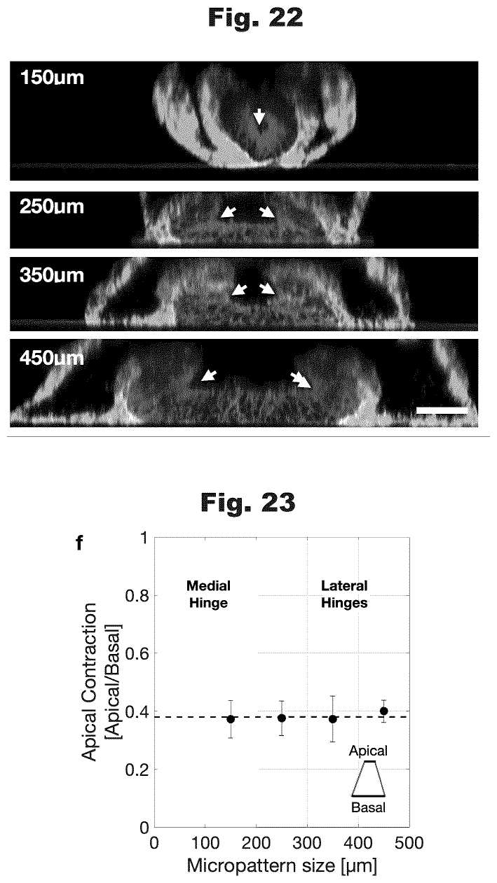

It was tested how tissue size controls shape by micropatterning rectangular geometries at increasing widths (FIG. 20). It was observed that the width of the neural plate region scales linearly with micropattern size, whereas the surface ectoderm is restricted to a fixed region of ˜150 μm at the boundary of the micropattern (FIG. 21). Surprisingly, neural fold morphology does not simply scale with increasing size (FIG. 22). Instead, narrow micropatterns (150 μm width), exhibit a u-shaped neural fold with a single central hinge point, characteristic of posterior regions. In contrast, wide micropattern (>150 μm width) carry two lateral hinges, characteristic of anterior regions. Notably, neural tissue apical contractility did not change as a function of tissue size, suggesting that the different morphologies do not arise from changes in cell behaviors (FIG. 23).

To better understand the conditions for the appearance of hinge points, a computational model was applied. It was found that a combination of apical neural contractility and basal adhesion between epidermal and neural tissues are sufficient for neural fold morphogenesis and hinge point formation in silico. Furthermore, each hinge point is characterized by a finite size. In cases where the neural tissue size is of order of the hinge point size, a single hinge point appears the center of the neural tissue, whereas for larger tissues two hinge points appears. Overall the data suggest a new scenario, in which a combination of tissue mechanics, patterning geometry, and cell behaviors determine the final shape of the neural tube.

### Modeling Neural Tube Defects

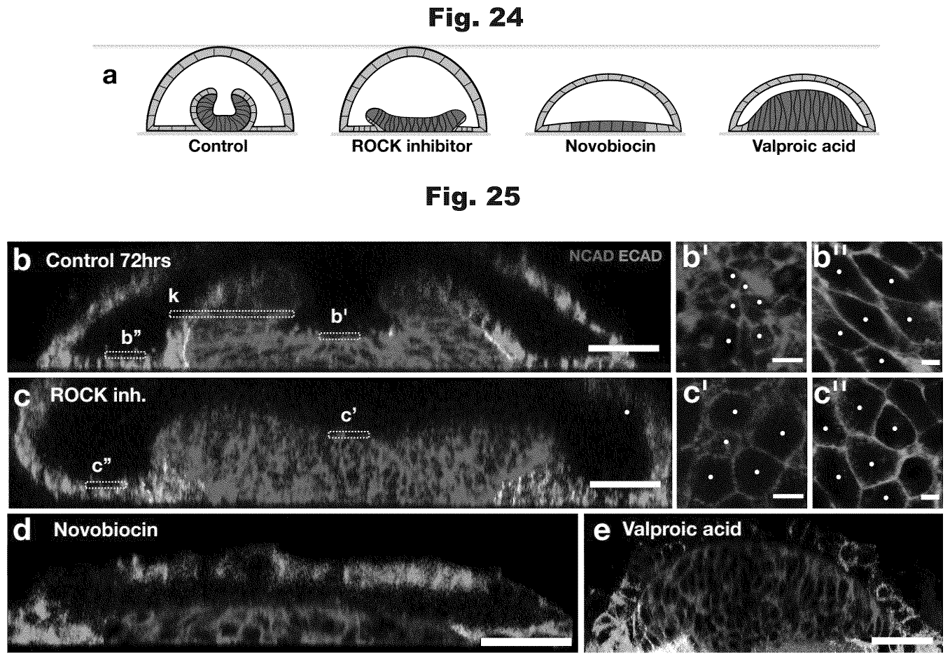

Because animal models are limited in their capacity to explain how human genetics affects neural tube closure, it was tested whether the reproducible human stem-cell system could be used to model neural tube defects (NTDs). This example focuses on various compounds that induce NTDs: ROCK inhibitor, novobiocin, and valproic acid (FIG. 24). In regards to pathophysiology of ROCK inhibitor, Shroom3 is upregulated in the neural tissue, and localizes ROCK to actin fibers in the apical surface of the neural plate. In turn, ROCK upregulates apical actomyosin contractility in neural cells which leads to apical constriction of the neural plate. Neural plate contraction then drives neural folding. It was thus set out to determine whether apical contractility plays a role during in vitro neural folding. It was observed that within 24 hrs after exposure to BMP the neural tissue exhibits high apical actomyosin levels, whereas the ectoderm tissue exhibits low apical actomyosin levels. The formation of an actomyosin gradient is followed by apical neural contraction which leads to folding. Thus, the data suggests that actomyosin dependent neural contractility plays an active role during in vitro neural folding, and is thus a good candidate to target for studying neural tube defects.

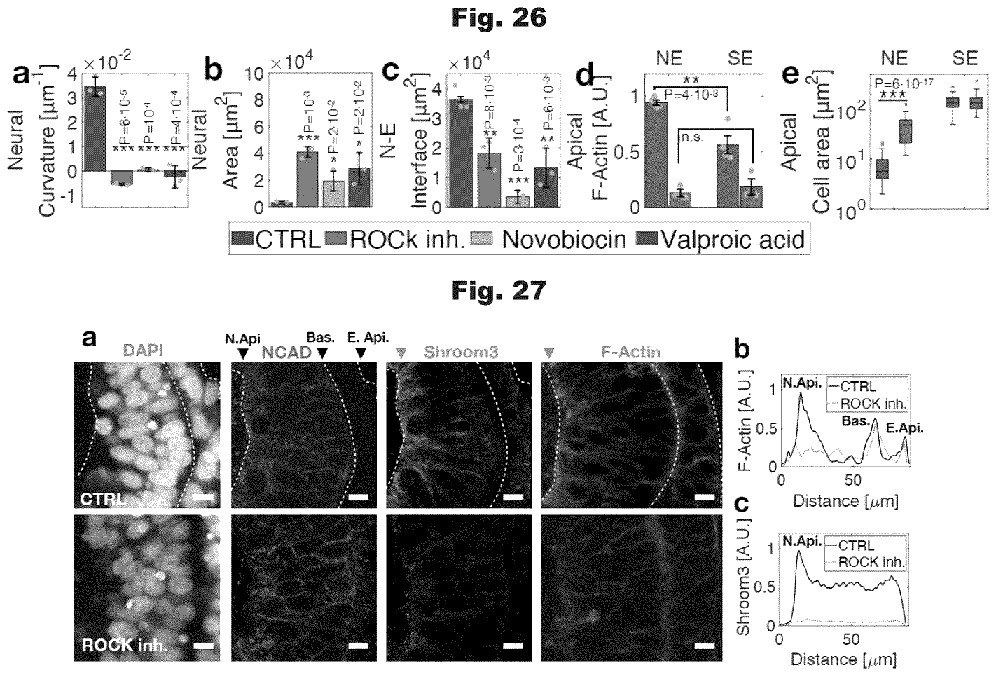

To perturb ROCK/Shroom3 signaling and apical neural contractility a highly specific molecular inhibitor of ROCK (Y-27632) was applied. ROCK inhibition results in severe folding defects similar to the open book morphology observed in embryonic cranial NTDs (FIG. 25). The effects of novobiocin and valproic acid were also examined. Samples treated with ROCK inhibitor, novobiocin and valproic acid exhibit a thick and flat neural tissue, which lacks neural hinges and is significantly less curved than control samples (FIG. 26). The neural apical cell areas and total apical tissue area are significantly larger in the treated samples, whereas epidermal cell areas do not exhibit a significant change (FIGS. 25 & 26). Furthermore, Shroom3 and F-actin localization to the apical neural surface are disrupted in response to ROCK inhibition, indicating that the assay perturbs shroom signaling, disrupts apical actomyosin assembly, and prevents apical contraction of the neural tissue (FIGS. 26 & 27). In contrast, actin localization to the basal surface of epidermal/neural tissues, as well as the epidermal apical surface, is not significantly perturbed. Taken together, these data demonstrate that interference with ROCK/Shroom3 signaling leads to perturbation of apical neural contractility and results in folding defects. This indicates that neural contractility is required for folding morphogenesis. It was further concluded that the in vitro lumen model system is suitable for studying how abnormalities in signaling pathways lead to neural tube defects in a human genetic context.

### Cardiac Lumen Formation and Differentiation

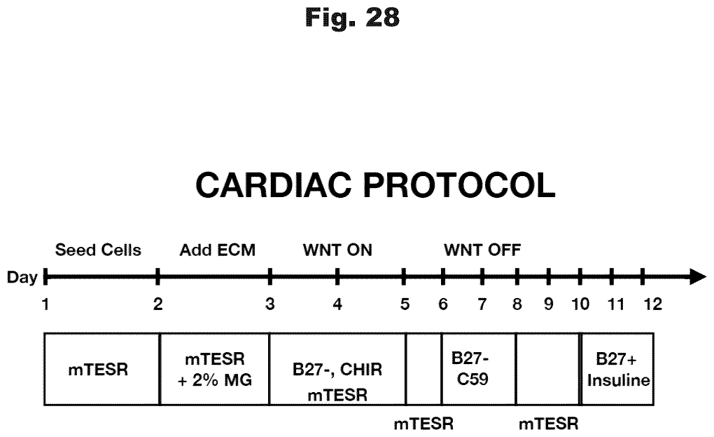

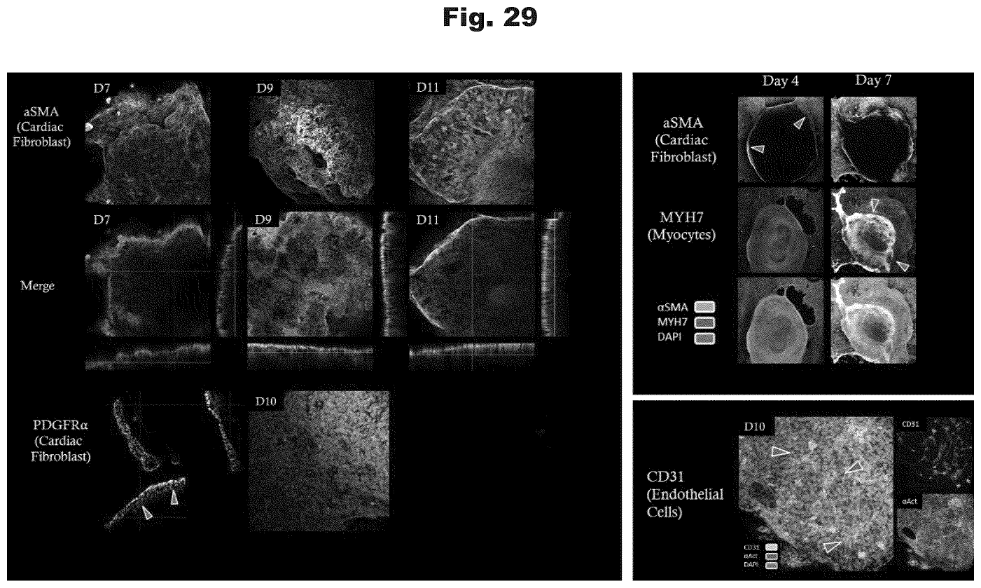

Provided in FIG. 28 is a protocol for generating cardiac organoids. Exemplary results of generated cardiac organoids are shown in FIG. 29.

### Methods Supporting Exemplary Embodiments

### Microfabrication and Soft Lithography

Device fabrication is performed using standard soft-lithography techniques on a four-inch wafer. One layer of photoresist (SU-8 2075, Microchem) is spun onto a silicon wafer at a thickness of 110 μm. Photoresist is exposed to ultraviolet light using a mask aligner (Suss MicroTec MA6) and unexposed photoresist is developed away to yield multiple arrays of posts. A Trimethylchlorosilane layer is vapor deposited on the developed wafer to prevent adhesion. A 10:1 ratio of PDMS and its curing agent (SYLGARD 184 A/B, Dow Corning) is poured onto the wafers and cured at 65 C overnight. The PDMS layer is then peeled off the silicon mold and individual stamps are cut out using a razor blade for future use.

### Glass Micropatterning

Sterile PDMS stamps and 35 mm diameter custom-made glass-bottomed culture dishes are plasma treated for 1 minute on high setting (PDC-32G, Harrick Plasma) to activate both surfaces. Stamps are pressed features-side to the glass surface and held in place. To passivate the glass surface in nonpatterned regions, 0.1 mg/mL PLL-g-PEG solution (SuSoS AG, Switzerland) is added to petri dish immediately after securing stamps to glass surface and incubated for 30 minutes. Stamps are then carefully removed and stamped glass dishes are rinsed several times with PBS++. Laminin-521 (STEMCELL Tech.) is added at a dilution of 5 μg/mL in PBS++ to incubate overnight at 4° C. The following day, stamped glass dishes are rinsed with PBS++ to remove excess unbound laminin and used within 1-7 days.

hPSC Lines and Maintenance

The work reported in this paper was approved by the Human Stem Cell Research Oversight Committee (hSCRO) at University of California Irvine, study #2018-1072. In this work, early passages of hiPSC was used. The cell line was previously karyotyped as normal. In addition, hiPSC reporter line AICS-0023 developed by the Allen institute and NIH-approved embryonic stem cell line RUES2 NIH approval number NIHhESC-09-0013 were used. hPSCs were cultured with mTESR1 media (STEMCELL Technologies, Cambridge, MA) on hESC-qualified Matrigel (Corning Lifesciences, Corning, NY) coated dishes. Media was exchanged on a daily basis, and cells were regularly checked for mycoplasma contamination.

### Neural Tube Morphogenesis Protocol

Day 1. Seeding onto micropatterns. hPSCs are released from well-plate surfaces using non-enzymatic agitation following manufacturer's instructions (ReleSR, STEMCELL Technologies, Cambridge, MA). Cells are resuspended as a single-cell suspension at densities of 750K-1M cells/mL in mTeSR1 containing 10 μM ROCK inhibitor Y27632 (Abcam, Cambridge, UK). 200 μL of cell suspension is then pipetted onto prepatterned dishes and allowed to settle for 15 minutes before adding 1 mL of mTeSR1 and allowing cells to settle for 10 additional minutes. Excess media is aspirated, leaving enough liquid to cover patterns and replaced with fresh 2 mL of mTeSR until the following day.

Day 2. Matrigel addition. mTESR1 media is exchanged with a neural induction media containing Matrigel (4%, v/v). Neural induction media is supplemented with 5 μM of TGFβ-inhibitor SB-431542.

Day 3-4. Lumen Formation. Dishes are left unperturbed at day 3 to allow transition into 3D stem-cell tissue containing a single lumen.

Day 5-9. Exposure to Morphogens. Neural induction media is supplemented with 5 ng/ml BMP4 in addition to 5 μM of SB-431542. Media is exchanged daily. Cell fates are observed on day 6 and folding is observed during days 7-9.

### Whole-Mount Immunostaining

All samples were fixed, immunostained and imaged as whole mounts in the culture dish. Samples are fixed in 4% PFA, 1 hr at room temperature (RT), washed three times for 15 min in PBS, and permeabilized in 1.5% Triton-PBS over-night (o/n) at 4 C. The following day, samples are washed in 0.3% Triton, and blocked for 2 hrs RT (10% NGS, 1% BSA, 0.3% Triton-X in PBS). Primary antibodies are used at 1:100-1:200 in blocking solution o/n at 4° C. Next, samples are washed in 0.1% Tween in PBS, and incubated with secondary antibodies 1:500-1:1000 in PBT o/n at 4° C. DAPI and phalloidin are also added at this stage. Finally, samples are washed in PBS for 1 hr, and imaged.

### ROCK Inhibition

ROCK inhibition is achieved using the small molecule Y-27632 reconstituted in water at a stock concentration of 10 mM. ROCK inhibitor was applied at a concentration of 10 μM (1:1000 dilution) at day 5, together with BMP, and maintained for 72 hrs until the end of the experiment at day 8. Media was changed daily. Control experiments were carried under identical conditions, adding water instead of ROCK inhibitor.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. A method of cellular luminal structure formation comprising:
providing a micropatterned matrix for supporting attachment of biological cells;
seeding a plurality of biological cells onto the matrix such that the plurality of biological cells forms a layer upon the micropatterned matrix; and
overlaying a medium onto the plurality of biological cells, the medium comprising suspended matrix.

2. The method of claim 1 wherein the overlaying the medium comprising suspended matrix results in a three-dimensional cellular luminal structure.

3. The method of claim 2 wherein the cellular luminal structure comprises a luminal floor upon the micropatterned matrix and a luminal wall that extends from the edges of the luminal floor.

4. The method of claim 2 wherein the luminal structure is dome-shaped or semi-tubular shaped.

5. The method of claim 4 wherein the shape of the luminal structure is dependent on the shape of the micropatterned matrix.

6. The method of claim 1 wherein the plurality of biological cells are epithelial cells.

7. The method of claim 1 wherein the plurality of biological cells expresses adhesion molecule proteins that result in an interconnected cell layer.

8. A system for cellular luminal structure formation comprising:
a micropatterned matrix on a substrate, the micropatterned matrix supporting attachment of biological cells;
a plurality of biological cells in layer upon the micropatterned matrix; and
a medium in contact with the plurality of biological cells, the medium comprising suspended matrix.

9. The system of claim 8 wherein the plurality of biological cells are epithelial cells.

10. The system of claim 9 wherein the epithelial cells are squamous, cuboidal, or columnar.

11. The system of claim 8 wherein the plurality of biological cells expresses adhesion molecule proteins that result in an interconnected cell layer.

12. The system of claim 8 wherein the plurality of biological cells are embryonic stem cells, induced pluripotent stem cells, neural stem cells, primary epithelial cells, intestinal epithelial cells, endothelial cells, primary endothelial cells, cardiac endothelial cells, pulmonary epithelial cells, pancreatic epithelial cells, gastric epithelial cells, renal epithelial cells, liver epithelial cells, neuroepithelial cells, or skin epithelial cells.

13. The system of claim 8 wherein the micropatterned matrix is collagen, laminin, fibronectin, elastin, alginate, poly-lysine, poly-arginine, polysaccharide, Matrigel, or Geltrex.

14. The system of claim 8 wherein the suspended matrix is collagen, laminin, fibronectin, elastin, alginate, poly-lysine, poly-arginine, polysaccharide, Matrigel, or Geltrex.

15. A cellular luminal structure formed in vitro, comprising:
a layer of interconnected biological cells upon a micropatterned matrix, wherein the layer of interconnected biological cells forms a luminal floor; and
a plurality of interconnected biological cells that extend from and are interconnected with the edges of the interconnected biological cells of the luminal floor to form a luminal wall such that the interconnected biological cells of the luminal floor and the interconnected biological cells of the luminal wall form a closed semi-tubular or dome-shaped lumen.

16. The cellular luminal structure of claim 15 further comprising a medium comprising suspended matrix, the medium in contact with the interconnected cells of the luminal wall.

17. The cellular luminal structure of claim 15 wherein the interconnected biological cells of the luminal floor and the interconnected biological cells of the luminal wall are epithelial cells.

18. The cellular luminal structure of claim 15 wherein the interconnected biological cells of the luminal floor and the interconnected biological cells of the luminal wall are embryonic stem cells, induced pluripotent stem cells, neural stem cells, primary epithelial cells, intestinal epithelial cells, endothelial cells, primary endothelial cells, cardiac endothelial cells, pulmonary epithelial cells, pancreatic epithelial cells, gastric epithelial cells, renal epithelial cells, liver epithelial cells, neuroepithelial cells, or skin epithelial cells.

19. A method of cellular differentiation comprising:
providing a cellular luminal structure formed in vitro, wherein the cells that form the cellular luminal structure are potent; and
feeding the cellular luminal structure, a differentiation induction medium and exposing the cellular luminal structure to morphogenic factors.

20. The method of claim 19, wherein the differentiation induction medium is a neural induction media, renal induction media, angiogenesis induction media, intestinal induction media, pancreatic induction media, lung induction media, vascular induction media, and skin induction media.
